(19) United States
(12) Patent Application Publication
(10) Pub. No.: US 2008/0077213 A1
(43) Pub. Date: Mar. 27, 2008

(54) Apparatuses and Methods for Adjusting Body Temperatures

(76) Inventor: Samuel C. Vickroy, Madison, AL (US)

Correspondence Address: LANIER FORD SHAVER & PAYNE P.C., P O BOX 2087, HUNTSVILLE, AL, 35804-2087, US

(21) Appl. No.: 11/859,399

(22) Filed: Sep. 21, 2007

Related U.S. Application Data
(60) Provisional application No. 60/846,301, filed on Sep. 21, 2006.

(51) Int. Cl. A61F 7/00

(52) U.S. Cl. 607/108

(57) ABSTRACT

A cooling device has a shell made up of a first layer and a second layer. The cooling device further has a medium transfer apparatus positioned between the first layer and the second layer that is detachably coupled to a medium injection apparatus. The medium injection apparatus contains a medium such that the medium is forced by the medium injection apparatus through the medium transfer apparatus thereby altering a temperature of the shell.

### CROSS REFERENCE TO RELATED APPLICATION

This Application claims priority to U.S. Provisional Patent Application No. 60/846,301, entitled “Apparatuses and Methods for Adjusting Body Temperatures” and filed on Sep. 21, 2006, which is incorporated herein by reference.

### RELATED ART

There are a number of reasons one might want to decrease the temperature of a body part. For example, during physical activity, a person's body temperature rises, and applying cool or cold temperatures to the body part reduces the temperature. The temperature reduction may enable the person to continue the physical activity and/or bring comfort to the person.

In addition, one may have an injury to a body part resulting from some type of physical activity. Oftentimes, it is advantageous to apply cool or cold temperatures to the injured body part in order to mitigate or eliminate pain associated with the injury or mitigate or eliminate swelling associated with the injury.

### SUMMARY

Embodiments of the present disclosure generally relate to apparatuses and methods for cooling. In particular, the present disclosure relates to apparatuses that are worn by a user to cool a body part.

An apparatus in accordance with an embodiment of the present disclosure comprises a shell having a first layer and a second layer and a medium transfer apparatus positioned between the first layer and the second layer of the shell. The medium transfer apparatus is detachably coupled to a medium injection apparatus containing a medium such that the medium is forced by the medium injection apparatus through the medium transfer apparatus thereby altering a temperature of the shell.

A method in accordance with an embodiment of the present disclosure comprises the step of detachably coupling a medium injection apparatus to a medium transfer apparatus that is positioned within a shell, the medium injection apparatus having a medium that is compressed at a pressure greater than atmospheric pressure. The method further comprises the step of injecting the medium from the medium injection apparatus through the medium transfer apparatus to the shell.

### BRIEF DESCRIPTION OF THE DRAWINGS

The disclosure can be better understood with reference to the following drawings. The elements of the drawings are not necessarily to scale relative to each other, emphasis instead being placed upon clearly illustrating the principles of the disclosure. Furthermore, like reference numerals designate corresponding parts throughout the several views.

FIG. 1 is a diagram illustrating a top view of a medium transfer apparatus in accordance with an exemplary embodiment of the present invention.

FIG. 2 is a diagram illustrating a cross-sectional view of the medium transfer apparatus depicted in FIG. 1.

FIG. 3 is diagram illustrating an exploded view of an exemplary shoe of the prior art.

FIG. 4 is diagram illustrating an exploded view of the shoe of FIG. 3 with the medium transfer apparatus of FIG. 1 inserted between an insole and an outsole of the shoe.

FIG. 5 is a diagram illustrating a cross-sectional view of the shoe depicted in FIG. 4.

FIG. 6 is a diagram illustrating a side view of the shoe depicted in FIG. 5 when a medium injection apparatus is being used to inject a medium through the medium transfer apparatus into the shoe.

FIG. 7 is a diagram illustrating the medium injection apparatus and a portion of the medium transfer apparatus depicted in FIG. 6.

FIG. 8 is a diagram illustrating a cross-sectional view of a shoe in accordance with an exemplary embodiment of the present disclosure.

FIG. 9 is a diagram illustrating a side view of a shoe having a built-in medium transfer apparatus in accordance with an exemplary embodiment of the present disclosure.

FIG. 10 is a diagram illustrating a front view of the shoe depicted in FIG. 9.

FIG. 11 is a diagram illustrating a top view of a shoe in accordance with an exemplary embodiment of the present disclosure.

FIG. 12 is a cross-sectional view of the shoe depicted in FIG. 11.

FIG. 13 is a flow chart illustrating a method for altering temperatures within a shoe in accordance with an exemplary embodiment of the present disclosure.

FIG. 14 is a flow chart illustrating a method for making a medium transfer apparatus in accordance with an exemplary embodiment of the present disclosure.

FIG. 15 is a diagram illustrating a wrist band having a medium transfer apparatus embedded therein in accordance with an exemplary embodiment of the present disclosure.

FIG. 16 is a diagram illustrating a cross-sectional view of the wrist band depicted by FIG. 15.

FIG. 17 is a diagram illustrating a cross-sectional view of the wrist band depicted by FIG. 15.

FIG. 18 is a diagram illustrating a top view of the medium transfer apparatus that is embedded in the wrist band depicted by FIG. 15.

FIG. 19 is a diagram illustrating a bottom view of the medium transfer apparatus that is embedded in the wrist band depicted by FIG. 15.

FIG. 20 is a diagram illustrating a fabric having a medium transfer apparatus embedded therein in accordance with an exemplary embodiment of the present disclosure.

FIG. 21 is a diagram illustrating a cross-sectional view of the fabric depicted by FIG. 20.

FIG. 22 is a diagram illustrating a hat having an inner region lined with the fabric depicted by FIG. 20.

FIG. 23 is a diagram illustrating a bottom view of the hat depicted in FIG. 22.

FIG. 24 is a diagram illustrating an exemplary ankle brace in accordance with an embodiment of the present disclosure.

FIG. 25 depicts a cross-sectional view of the ankle brace depicted by FIG. 24.

FIG. 26 depicts a diagram illustrating an outside layer of a medium transfer apparatus used in the ankle brace depicted in FIG. 24.

FIG. 27 depicts a diagram illustrating an inside layer of the medium transfer apparatus used in the ankle brace depicted in FIG. 24.

FIG. 28 is a diagram illustrating an exemplary knee brace in accordance with an embodiment of the present disclosure.

FIG. 29 depicts a cross-sectional view of the knee brace depicted by FIG. 28.

FIG. 30 depicts a diagram illustrating an outside layer of a medium transfer apparatus used in the knee brace depicted in FIG. 28.

FIG. 31 depicts a diagram illustrating an inside layer of the medium transfer apparatus used in the knee brace depicted in FIG. 28.

FIG. 32 is a diagram illustrating an exemplary elbow brace in accordance with an embodiment of the present disclosure.

FIG. 33 depicts a cross-sectional view of the elbow brace depicted by FIG. 32.

FIG. 34 depicts a diagram illustrating an outside layer of a medium transfer apparatus used in the elbow brace depicted in FIG. 32.

FIG. 35 depicts a diagram illustrating an inside layer of the medium transfer apparatus used in the elbow brace depicted in FIG. 32.

FIG. 36 is a diagram illustrating an exemplary neck brace in accordance with an embodiment of the present disclosure.

### DETAILED DESCRIPTION

The present disclosure generally pertains to apparatuses and methods for cooling various body parts and/or extremities. In accordance with one exemplary embodiment of the present disclosure, a medium transfer apparatus is inserted into and/or coupled to an article of clothing, such as, for example, a shoe, a wristband, a hat, or the like. The apparatus has an inlet that can be detachably coupled to a medium injection apparatus, such as a conventional spray can, that injects compressed air or some other cooling medium through the medium transfer apparatus into an interior region of the article of clothing. Thus, by injecting the cooling medium into the article of clothing, a user is able to cool an extremity without removing the article of clothing.

In one embodiment, the cooling medium is directed to an insole of a shoe such that the insole is significantly cooled by the cooling medium being injected into the shoe. The cooled insole continues to draw heat from the user's foot and the user continues to feel a cooling sensation long after the cooling medium injection has ended.

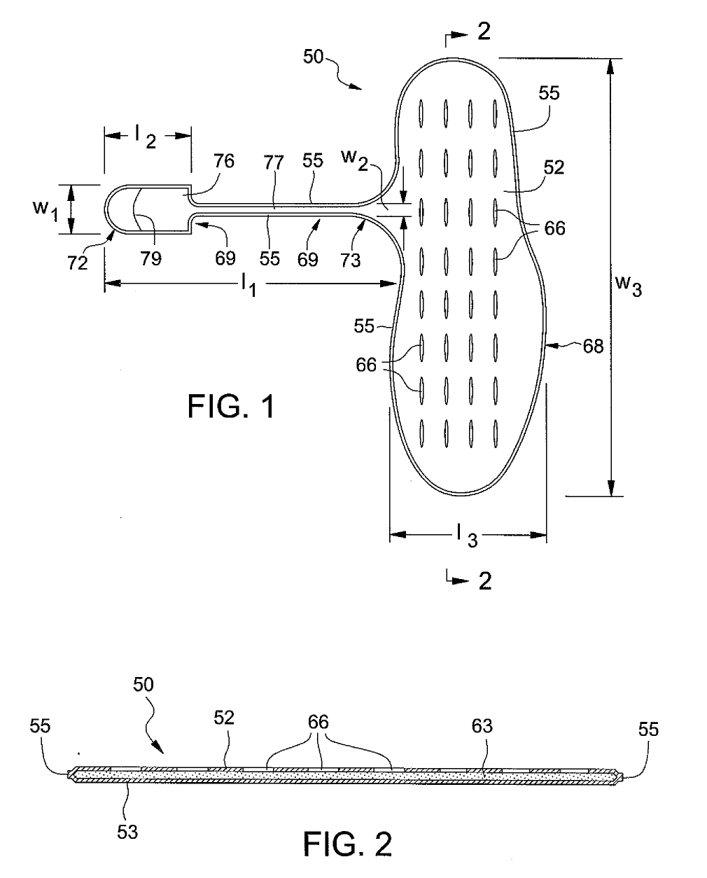

FIGS. 1 and 2 depict a medium transfer apparatus 50 in accordance with an exemplary embodiment of the present disclosure. The medium transfer apparatus 50 comprises an upper layer 52 that has been melded with a lower layer 53 to form a bead 55 joining the upper and lower layers 52 and 53, which form a foot-shaped base 68 and an inlet 69. The upper and lower layers 52 and 53, as well as the bead 55, form a cavity 63 that is airtight except for a plurality of apertures 66 in the upper layer 52 and except for the inlet 69. As will be described in more detail hereafter, the inlet 69 allows a medium, such as cooled or heated air, to enter the cavity 63, and the apertures 66 allow the medium to exit the cavity 63.

In the embodiment depicted by FIG. 1, the inlet 69 is hollow and open at both ends 72 and 73 such that a medium may be injected into the end 72 and pass into the cavity 63 through end 73. The end 72 of the inlet 69 forms a tab 76 that is wider than a throat 77 of the inlet 69, as shown by FIG. 1. In one embodiment, the width (w₁) of the tab 76 is about 1¼ inches, and the width (w₂) of the throat 77 is about one-quarter of an inch, although w₂ increases slightly at end 73 making this end 73 tapered. By being wider than the throat 77, the tab 76 is easier to locate and grasp by a user. In addition, a length (l₁) of the inlet 69 is about 5⅛ inches, and a length (l₂) of the tab 76 is about 1½ inches. In other embodiments, other shapes and dimensions of the inlet 69 are possible.

As shown by FIG. 1, the tab 76 has a slit 79 in its upper layer 52. The slit 79 provides an opening through which a medium can be injected, as will be described hereafter in more detail.

A width (w₃) of the base 68 is about 10 inches, and a length (l₃) of the base 68 is about 2½ inches. In FIG. 1, the perimeter of the base 68 is generally shaped like a foot in two dimensions (2D). However, other shapes and dimensions of the base 68 are possible in other embodiments.

The bead 55 forms the outer perimeter of both the base 68 and inlet 69. Further, each layer 52 and 53 is composed of polyvinyl chloride (PVC) and is about 0.006 inches thick. In addition, the material of the apparatus 50 is flexible. However, in other embodiments, other types of material, including flexible and/or inflexible material, as well as porous and/or non-porous, may be used, and other dimensions are possible.

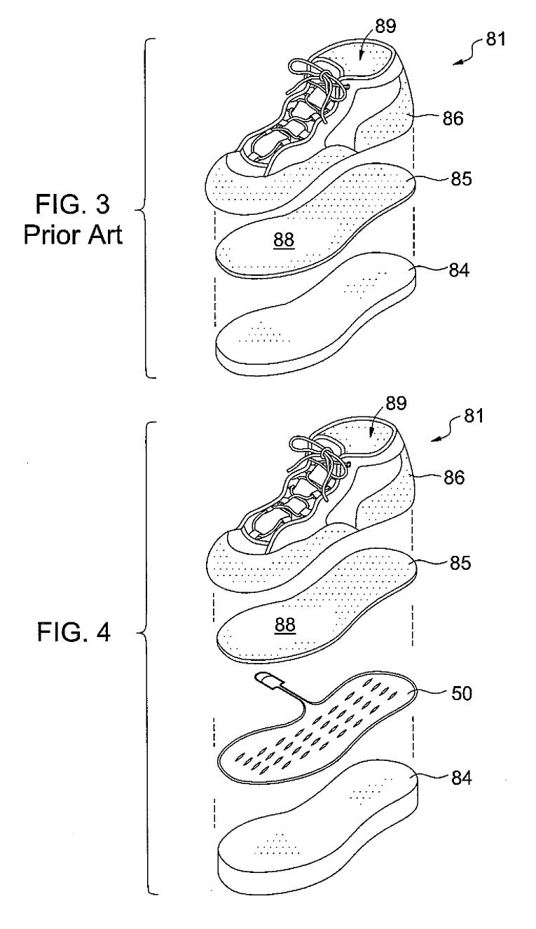

In one embodiment, the apparatus 50 is inserted into a shoe, and the inlet 69 is positioned such that end 72 is exposed and conveniently accessible to a user. FIG. 3 depicts an exemplary shoe 81 of the prior art. The shoe 81 comprises an outsole 84, an insole 85, and a vamp 86. The outsole 84 is attached to the vamp 86, and the insole 85 resides between the vamp 86 and the outsole 84. When a user is wearing the shoe 81, the bottom of the user's foot is pressed against the upper surface 88 of the insole 85 such that the user's weight is supported by the outsole 84 and insole 85 with the vamp 86 covering the top of the user's foot. Although the insole 85 is inserted between the vamp 86 and the outsole 84, the insole 85 is often not fixedly attached to either the vamp 86 or the outsole 84 so that the insole 85 can be removed through an opening 89 in the vamp 86.

In one exemplary embodiment, the base 68 of the medium transfer apparatus 50 is positioned between the outsole 84 and the insole 85, as depicted by FIG. 4. For example, the insole 85 may be removed from the shoe 81 through the opening 89, and the medium transfer apparatus 50 may be inserted into the shoe 81 through this same opening 89. In particular, the apparatus 50 may be positioned on the upper surface of the outsole 84, and the insole 85 may then be re-inserted into the shoe 81 through the opening 89 such that a bottom surface of the insole 85 contacts the upper layer 52 of the apparatus 50. Thus, a medium passing through the apertures 66 in the layer 52 strikes the bottom surface of the insole 85, as will be described in more detail hereafter.

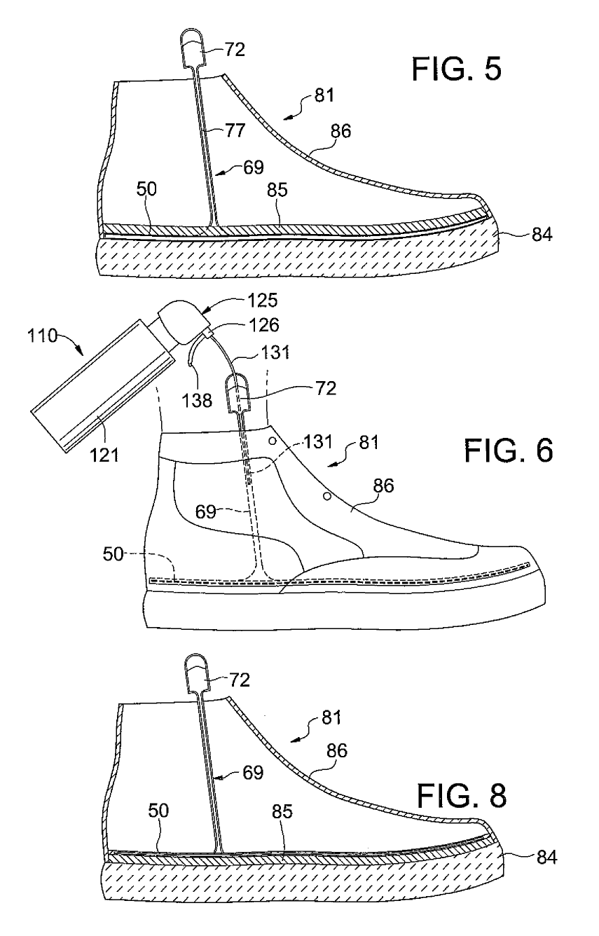

FIG. 5 depicts a cross-sectional view of the shoe 81 after the apparatus 50 has been inserted as described above. In the embodiment depicted by FIG. 5, the inlet 69 has been positioned such that it passes through opening 89, and the end 72 of the inlet 69 is exposed. In other embodiments, the inlet 69 may be positioned differently. When a person's foot is inserted into the shoe 81, as shown by FIG. 6, the person's foot, ankle, and/or leg presses the inlet 69 against an inner surface of the vamp 86 thereby keeping the inlet 69 in the same approximate position such that the end 72 remains exposed and accessible.

When desired, a user may cool his foot by interfacing a medium injection apparatus 110 with the exposed end 72 of inlet 69 and then using the apparatus 110 to inject a cooling medium, such as compressed air or oxygen, into the cavity 63 of the medium transfer apparatus 50. The cooling medium is forced through the apertures 66 in the upper layer 52 of the apparatus 50 and strikes the insole 85 lowering the temperature of the insole 85. Lowering the temperature of the insole 85 helps to draw heat from the user's foot thereby cooling the user's foot. Further, some of the cooling medium may be forced around the edges of the insole 85 into the region between the vamp 86 and the insole 85. Such medium may strike the user's foot further cooling the user's foot. Moreover, injecting the cooling medium into the shoe 81 may provide the user with an immediate cooling sensation as well as significantly lower the temperature of the insole 85 for a relatively long period of time (e.g., several minutes) so that the user continues to feel a cooling sensation long after the injection.

The material of the apparatus 50 is flexible in at least one embodiment. In such an embodiment, there may be no space between the upper and lower layers 52 and 53 when medium is not being injected into the apparatus 50. When medium is injected into the apparatus 50, the medium will slightly separate portions of the upper layer 52 from the lower layer 53 as the medium travels through the apparatus 50.

In one exemplary embodiment, as shown by FIG. 6, the medium injection apparatus 110 comprises a spray can 121 having a nozzle 125. The cooling medium is contained within the spray can 121 and is under a pressure that is greater than atmospheric pressure. For example, in one embodiment, the cooling medium is under a pressure of about 160-180 pounds per square inch (psi) when measured at an ambient temperature of 130 degrees F. Many conventional spray cans of compressed air or other media are manufactured with contents at about 100 to 200 psi when measured at an ambient temperature of 130 degrees F., and any known or future-developed spray can be used to implement the medium injection apparatus 110. Such spray cans often include a refrigerant. In one embodiment, the cooling medium contained within the apparatus 110 is composed, at least partially, of difluoroxthane. For example, the medium in the apparatus 110 may be a compressed mixture of air and difluoroxthane. Further, other types of devices and other pressures are also possible.

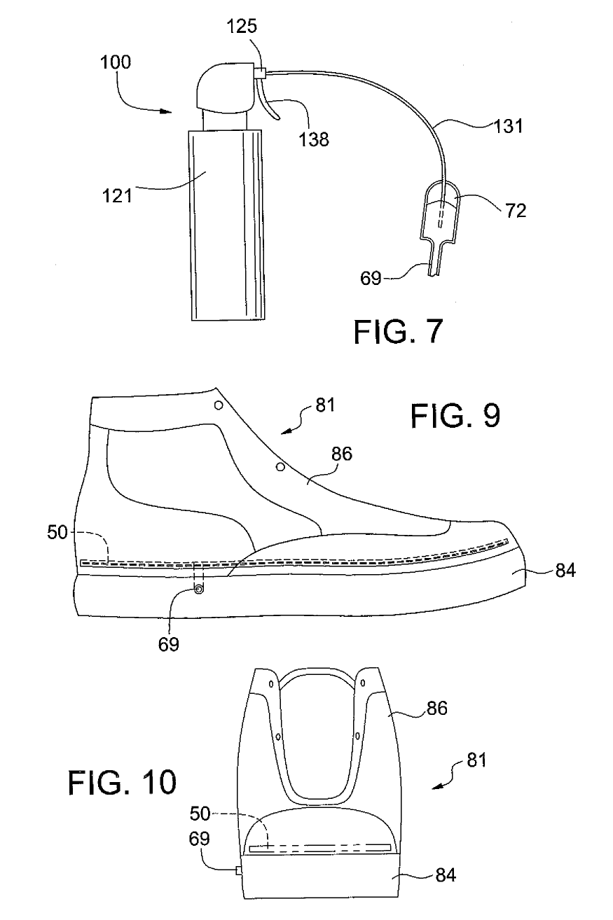

In the embodiment depicted by FIG. 6, the apparatus 110 comprises a removable hollow straw 131 that detachably couples the nozzle 125 to the end 72 of the inlet 69. The tip of the straw 131 and the inlet 69 are dimensioned such that the inlet 69 fits around an outer periphery of the straw tip, as shown by FIGS. 6 and 7. Various other techniques for interfacing the apparatus 110 and the inlet 69 are possible in other embodiments. Indeed, the use of a straw 131 is unnecessary, and the inlet 69 may be detachably coupled to the nozzle 125 in some other manner. For example, the nozzle 125 may be interfaced directly with the inlet 69.

In the instant embodiment, the cooling medium exiting the can 121 passes through a hollow tip 126 of the nozzle 125. The inner wall of the tip 126 fits snugly around the straw 131 such that frictional forces help to keep the straw 131 coupled to the tip 126 and such that medium exiting through the nozzle 125 preferably does not escape between the straw 131 and tip 126. In another example, the inner wall of the straw 131 may be dimensioned to snugly fit around the tip 126.

In addition, the width of the throat 77 is slightly larger than that of the straw 131 such that the straw 131 can be inserted into the inlet 69 through the slit 79 and pass into the throat 77, as shown by FIGS. 6 and 7. When a medium is being injected by the apparatus 110, a significant portion of the medium is forced through the throat 77 into the cavity 63 and then through the apertures 66, as will be described in more detail hereafter. In general, the closer that the straw 131 is inserted to the tapered end 73, the less likely it is that portions of the injected medium will escape through the slit 79.

In the embodiment shown by FIG. 6, the nozzle 125 comprises a trigger 138 that when actuated by a user, releases the cooling medium within the can 121 through the nozzle 125. In this regard, actuation of the trigger 138 transitions the nozzle 125 to a state in which a path is provided from the interior of the can 121 through the nozzle 125 to the atmosphere or, in the case when the nozzle 125 is interfaced with the inlet 69, to the cavity 63 of the apparatus 50. Since the contents of the can 121 are under pressure greater than the external pressure, the cooling medium of the can 121 is expelled through such a path when the trigger 138 is actuated. When the trigger 138 is released or otherwise no longer actuated, the path is closed such that the contents of the can 121 are not allowed to escape. The operation of the spray can 121 described herein may be similar or identical to that of other conventional spray cans and other types of medium injection apparatuses.

In one exemplary embodiment, the cooling medium that is within the apparatus 110 and injected into the shoe 81 by the apparatus 110 is compressed air. The temperature of the cooling medium compressed at about 160-180 psi can be very low, such as close to or below freezing (ie., 32 degrees F.) upon exiting the spray can 121. Thus, the cooling medium being injected into the shoe 81 in the instant example is likely significantly colder than the interior region of the shoe 81 prior to the injection. Accordingly, injection of the cooling medium within the shoe 81, as described herein, has a significant effect to the temperatures within the shoe 81 and provides the user with a significant cooling sensation.

In fact, injecting the cooling medium into the shoe 81, as described above, for only a short time, such as a few seconds, can have a significant and prolonged impact to the temperatures within the shoe 81. In particular, the insole 85, which is struck directly by the cooling medium in the instant example, can be significantly cooled such that its temperature remains significantly below its original temperature (i.e., its temperate prior to the cooling medium injection) for a prolonged period (e.g., about ten minutes or more) without another injection. Accordingly, an athlete, such as a tennis or football player, may make an injection during a short break in play, yet the effects of the injection may linger well after play has resumed.

FIG. 8 depicts another embodiment in which the medium transfer apparatus 50 is positioned on the upper surface 88 of the insole 85 rather than between the insole 85 and the outsole 84. In this regard, the upper layer 52 faces the insole surface 88 such that the cooling medium passing through the apertures 66 strikes and cools the insole surface 88. However, the presence of the apparatus 50 between the user's foot and the insole 85 may somewhat shield the insole 85 from the foot. Depending on the heat transfer characteristics of the insole 85 and the apparatus 50, the insole 85 may be better cooled and/or cooled for a longer time period relative to the apparatus 50 such that it would be more preferable for the user's foot to abut the insole 85, as described above for the embodiment depicted in FIG. 5. However, the injected cooling medium cools the apparatus 50 as well as the insole 85, and if desired, the apparatus 50 may abut the user's foot so that heat is drawn from the user's foot by the apparatus 50.

In another exemplary embodiment, the medium transfer apparatus 50 is positioned on the upper surface 88 of the insole 85, similar to the embodiment depicted by FIG. 8. However, the lower layer 53 faces the insole 85 such that the upper layer 52 faces the user's foot. Thus, the cooling medium passing through the apertures 66 directly strikes the user's foot. In such an embodiment, it is not likely that the insole 85 is as affected by the injection as in the embodiments described above, possibly limiting the duration of the injection's effect to the insole 85. In addition, as described above, the temperature of the cooling medium can be very cold, such as close to or below freezing. In such an example, longer bursts of the cooling medium may not be possible without harming the user's foot. Thus, shorter bursts may be desired in order to prevent injury. Such shorter bursts, although giving the user an immediate cooling sensation, may have shorter lasting effects.

As described above, the medium transfer apparatus 50 may be inserted into the shoe 81 after the shoe 81 has been manufactured, such as by removing the insole 85, inserting the apparatus 50, and inserting the insole 85 back into the shoe 81. In other embodiments, the apparatus 50 may be built-in such that it is inserted into the shoe 81 during manufacturing. For example, FIGS. 9 and 10 depict an embodiment in which the apparatus 50 is positioned in the shoe 81 during manufacturing, and the inlet 69 passes through the outsole 84. In this regard, as best illustrated in FIG. 10, an end 72 of the inlet 69 protrudes from the outsole 84. In the depicted embodiment, the end 72 is not wider than the throat 77. An inner periphery of the end 72 can, however, be about the same as the outer periphery of the straw 131 such that the end 72 snugly fits around the straw 131 when the straw 131 is interfaced with the end 72. In another example, the end 72 may form a tab 72 that is wider than the throat 77, as described above. Except for the positioning of the inlet 69, the configuration of the shoe 81 and the apparatus 50 may be identical to any of the embodiments previously described above. In the embodiment depicted by FIGS. 9 and 10, the outsole 84 may be manufactured with a groove (not shown) through which the inlet 69 may be inserted when the apparatus 50 is positioned during manufacturing. In other embodiments, the inlet 69 may pass through other components of a shoe. For example, the inlet 69 may pass through a hole in the vamp 86.

In addition, in several of the embodiments described above, the width of the straw 131 has been described as being smaller than the width of the inlet 69 and, in particular, the throat 77 of the inlet 69. However, it is possible for the width of the straw 131 to be greater than the width of the inlet 69. As an example, the end 72 of the inlet 69 could be composed of a rigid material having an outer periphery slightly smaller than the inner periphery of the straw 131. In such an embodiment, the medium injection apparatus 110 could be interfaced with the medium transfer apparatus 50 by inserting the end 72 into the straw 131 such that the straw 131 fits snugly around the end 72. Various other techniques for interfacing the medium transfer apparatus 50 with the medium injection apparatus 50 would be apparent to one of ordinary skill in the art upon reading this disclosure.

In some embodiments described above, the medium transfer apparatus 50 is composed of flexible material. However, the apparatus 50 may be composed of inflexible material or a combination of flexible and inflexible material. For example, the inlet 69 may be composed of a rigid material, such as a rigid plastic material, and the base 68 may be composed of a flexible material. In another example, the end 72 may be covered by a hollow and rigid tip (not shown). Such a rigid tip may facilitate interfacing of the apparatuses 50 and 110.

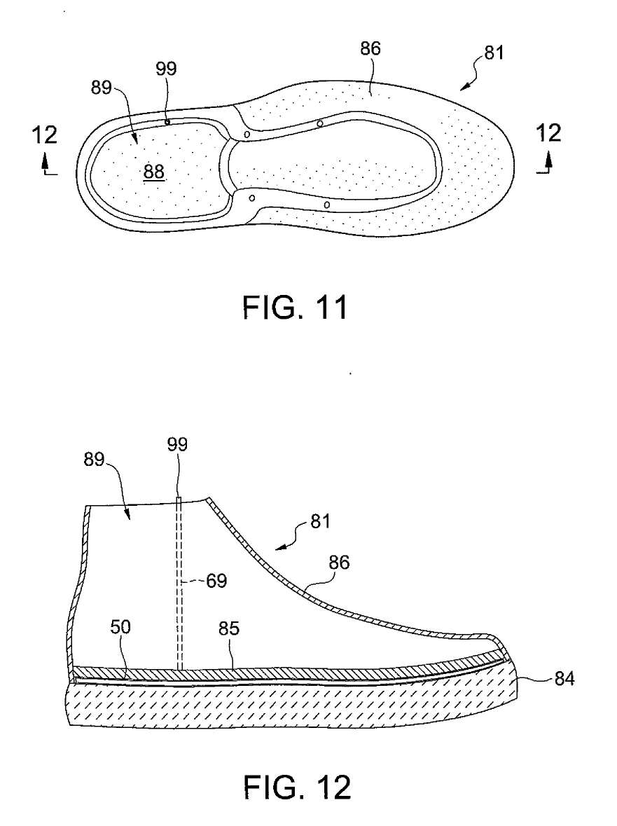

FIGS. 11 and 12 depict a shoe 81 in accordance with an exemplary embodiment of the present disclosure. The embodiment shown by FIGS. 11 and 12 is identical to that shown by FIG. 5 except that the inlet 69 is embedded in the vamp 86 and has a rigid tip 99 at end 72. The straw 131 may be interfaced with the tip 99 so that the cooling medium can be injected via inlet 69 into the cavity 63 of the apparatus 50 similar to the embodiments previously described above. The tip 99 is hollow and dimensioned such that the straw 131 snugly fits around the tip 99 or vice versa. For example, the outer periphery of the tip 99 may be about the same as the inner periphery of the straw 131 so that the straw 131 fits snugly around the tip 99, or the outer periphery of the straw 131 may be about the same as the inner periphery of the tip 99 so that the tip 99 fits snugly around the straw 131.

Note that the apparatus 50 may be positioned differently in other yet embodiments. For example, the base 68 of the apparatus 50 may be embedded within the insole 85. In such an embodiment, the insole 85 may have a hole (not shown) for allowing the inlet 69 to pass out of the insole 85, or the inlet 69 may be an integral component of the insole 85. If the inlet 69 passes through a hole in the insole 85, such a hole may be large enough to allow the injected medium to exit the insole 85, or the insole 85 may have additional holes for allowing the injected medium to exit. In addition, the layers 52 and 53 may be composed of a material similar to that of the insole 85 so that the apparatus 50 can replace the conventional insole 85 or be used in lieu of the conventional insole 85.

It should be emphasized that in any of the embodiments described above, the nozzle 125 may be interfaced directly with the medium transfer apparatus 50 without the use of a straw 131. For example, in the embodiment depicted by FIGS. 11 and 12, the shoe tip 99 may fit snugly into nozzle tip 126 or vice versa. As an example, the outer periphery of the shoe tip 99 may be about the same as the inner periphery of the nozzle tip 126 so that the nozzle tip 126 fits snugly around the shoe tip 99, or the outer periphery of nozzle the tip 126 may be about the same as the inner periphery of the shoe tip 99 so that the shoe tip 99 fits snugly around the nozzle tip 126.

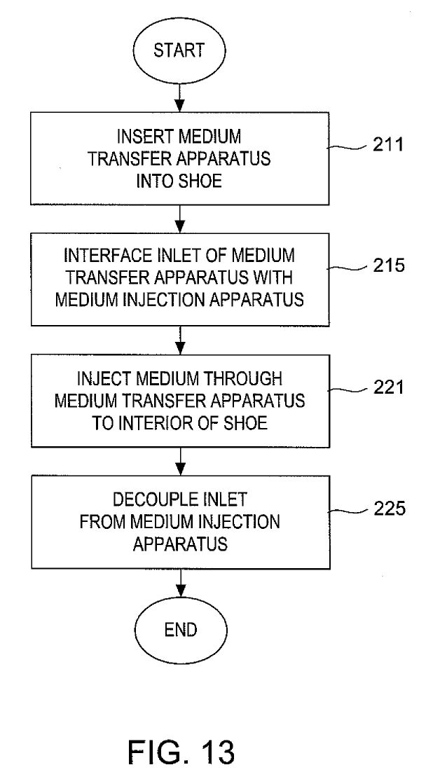

An exemplary use and operation of a medium transfer apparatus 50 will be described in detail hereinbelow with particular reference to FIG. 13.

Assume that a user of the shoe 81 shown by FIG. 3 desires to use the medium transfer apparatus 50 to cool his feet when wearing the shoe 81. Before donning the shoe 81, the user removes the insole 85 and inserts the medium transfer apparatus 50 into the shoe 81, as shown by block 211 of FIG. 13. The user then inserts the insole 85 back into the shoe 81 on top of the apparatus 50, as shown by FIGS. 4 and 5. In other examples, the apparatus 50 may be inserted and/or attached to the shoe 81 by the shoe manufacturer such that it is unnecessary for the user to insert the apparatus 50 into the shoe 81.

At some point, the user may desire to use the apparatus 50 for cooling his foot. For example, the user may be an athlete, such as a football player, and desire to use the apparatus 50 for cooling his foot after coming to the sideline during a football game. In another example, the user may be a tennis player and desire to use the apparatus 50 to cool his foot during a break between sets.

To cool his foot, the user interfaces the medium injection apparatus 110 with the inlet 69 so that the cooling medium in the apparatus 110 can be injected into the medium transfer apparatus 50 through the inlet 69, as shown by block 215 of FIG. 13. For illustrative purposes, assume that the apparatus 110 comprises a spray can 121, as described above with reference to FIG. 6, and that the cooling medium is composed of compressed air and a refrigerant, although other types of medium injection apparatuses 110 and cooling media may be used in other examples. In the instant example, the user couples one end of the hollow straw 131 to the nozzle 125 such that any of the cooling medium exiting the apparatus 110 passes through the straw 131. The user also inserts the opposite end of the straw 131 into the inlet 69 through the slit 79 such that the cooling medium passing through the straw 131 enters the inlet 69 and passes into the cavity 63 of the apparatus 50.

The user then operates the medium injection apparatus 110 such that cooling medium within the apparatus 110 is injected into the medium transfer apparatus 50, as shown by block 221 of FIG. 13. In the instant example, the user actuates the trigger 138 such that the cooling medium, which is under pressure, is forced out of the apparatus 110 through the nozzle 125. During injection, the relatively high pressure within the apparatus 110 forces the cooling medium through the straw 131 and inlet 69 into the cavity 63. In addition, such pressure also forces the cooling medium out of the cavity 63 through the apertures 66 such that the cooling medium strikes the insole 85 thereby cooling the insole 85. In the instant example, the cooling medium is pressurized to about 160-180 psi when measured at an ambient temperature of 130 degrees F., although other pressures are possible in other examples.

The duration of the injection may be for any time period, and the user may make more than one injection, if desired. Generally, the longer that cooling medium is injected into the shoe 81 via apparatus 50, the greater is the cooling effect to the user's foot and to the insole 85. In one example, the user maintains an injection for about 5 to 10 seconds. Such a duration, at the exemplary pressures described above for the instant example, can provide a significant cooling effect. Indeed, the insole 85 may remain below its original temperature (i.e., its temperature prior to the injection) for several minutes after the injection has ended. Moreover, the insole 85 may continue cooling the user's foot long after the injection has ended. Eventually, heat from the user's foot and/or other sources may raise the temperature of the insole 85 back to its original temperature, but at any time, the user may perform another injection to again cool his foot and the insole 85, as described above.

After performing at least one injection, the user decouples the medium injection apparatus 110 from the medium transfer apparatus 50, as shown by block 225, so that the medium injection apparatus 110 does not interfere with the user's activities. In the instant example, the user can decouple the medium injection apparatus 110 from the medium transfer apparatus 50 by simply pulling the straw 131 out of the inlet 69.

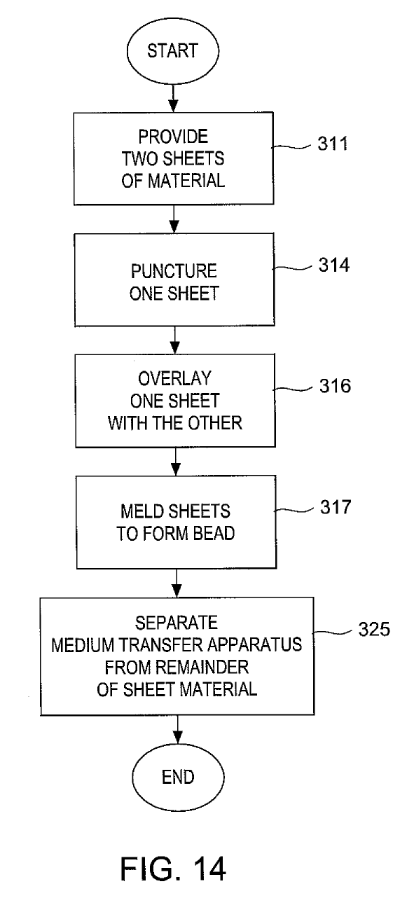

There are various methods that can be used to manufacture the medium injection apparatus 50. One exemplary method will be described in more detail hereinbelow with particular reference to FIG. 14.

Initially, two sheets of material for forming the layers 52 and 53 are provided, as shown by block 311 of FIG. 14. In one example, each of the sheets is composed of PVC and is about 0.006 inches thick, although other types of material and other thicknesses are possible in other examples. One of the sheets is punctured to form apertures 66, as shown by block 314 of FIG. 14. Any known or future-developed process of puncturing a sheet of material may be used.

One of the sheets is then overlaid with the other sheet, such that one of the sheets is positioned on top of the other sheet, as shown by block 316 of FIG. 14. The two sheets are then melded to form the bead 55, as shown by block 317 of FIG. 14. In one embodiment, a thermoformer is used to meld the two sheets in block 317. A thermoformer is a well-known press that uses radio frequency (RF) sealing for forming a seal. Such a seal can be of just about any desired pattern. In the instant example, a pattern is selected such that the perimeter of the bead portion forming the base 68 is in the general shape of a foot outline, as shown by FIG. 1, although other types of shapes are possible in other examples.

Once the bead 55 has been formed, the bead 55 and the sheet material within the perimeter of the bead 55 are separated from the remainder of the sheet material to provide the apparatus 50 depicted in FIG. 1, as shown by block 325 of FIG. 14. In this regard, the portion of the sheet material forming the apparatus 50 can be easily separated from the remainder of the sheet material by hand, although some type of automated separating process may be used instead.

The above embodiments have been described as injecting a medium for cooling a user's foot. In other examples, the injection medium apparatus 110 may contain a heated medium, which can be injected into the shoe 81 according to the same or similar techniques described above for injecting a cooling medium so that the user's foot and/or the insole 85 can be warmed instead of cooled.

Additionally, it should be emphasized that the aforedescribed embodiments of the medium transfer apparatus 50 and the medium injection apparatus 110 are exemplary, and other configurations of the apparatuses 50 and 110 are possible without departing from the principles of the present disclosure. In addition, the shoe 81 depicted above is also exemplary, and apparatuses 50 and 110 may be used with other types of known or future-developed shoes in other embodiments. Further, the method of manufacturing an exemplary medium transfer apparatus 50 is described above for illustrative purposes, and other methods of manufacturing a medium transfer apparatus 50 are possible.

Furthermore, it should be noted that the medium transfer apparatus 50 can be used in locations other than in shoes to allow the apparatus 50 to cool or heat parts of the body other than feet. For example, the apparatus 50 may be embedded within clothing fabric and used to alter the temperatures of such fabric.

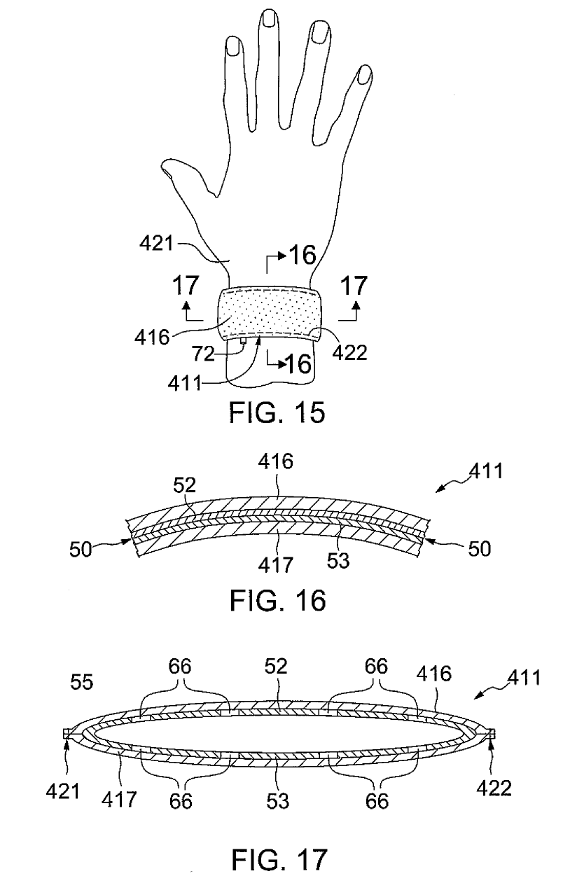

FIG. 15 depicts an exemplary wrist band 411 being worn on a wrist of a user. As shown by FIGS. 15-17, the wrist band 411 comprises an upper layer 416 and a lower layer 417 of fabric, such as cotton, forming a shell. The apparatus 50 is disposed between the layers 416 and 417 so that the apparatus 50 is embedded within the wrist band 411. Note that the layers 416 and 417 are sewn together along seams 421 and 422.

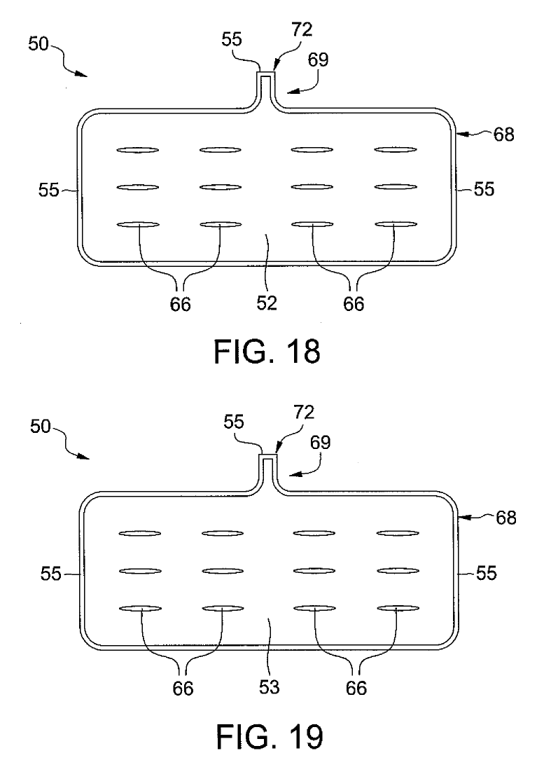

As shown by FIG. 18, the apparatus 50 of the instant embodiment is similar to the apparatus 50 of the embodiment depicted by FIG. 1. However, the base 68 is dimensioned such that it fits between the layers 416 and 417. Further, as in the previously described embodiments, the apparatus 50 of FIG. 18 has an inlet 69 that allows a medium from a medium injection apparatus 110 to be injected into the cavity 63 of the apparatus 50. As shown by FIG. 15, the end 72 of the inlet 69 is exposed.

As shown by FIGS. 17-19, apertures 66 exist in both layers 52 and 53 of the apparatus 50. Thus, when a cooling medium is injected into the apparatus 50, the medium exits the apertures 66 in both layers 52 and 53 and strikes both of the fabric layers 416 and 417. Thus, both of the fabric layers 416 and 417 are cooled. Note that FIG. 16 shows no space between layers 52 and 53, such as may be the case when the cooling medium is not being injected into the apparatus 50. FIG. 17, however, shows a slight separation between the layers 52 and 53, such as may be the case when the cooling medium is being injected into the apparatus 50.

To provide an enhanced cooling sensation, it is generally desirable for the fabric layer in contact with the user's body to be cooled by the cooling medium injection. In the instant example, the bottom fabric layer 417 contacts the user's wrist, and the apertures 66 in the bottom layer 53 of the apparatus 50 allow the cooling medium to strike this fabric layer 417 thereby enhancing the cooling sensation to the user.

Moreover, the upper fabric layer 416 is not in direct contact with the user's body in FIG. 15. If desired, the upper layer 52 could be configured without apertures 66 such that all of the cooling medium is directed through the apertures 66 in layer 53 toward the fabric layer 417 is in direct contact with the user's wrist. However, the user may rub the upper fabric layer 416 against parts of his body (e.g., the user may rub the layer 416 against his forehead or face to wipe perspiration from his forehead or face). By configuring the layer 52 with apertures 66, cooling medium injected through the apparatus 50 is directed toward the upper fabric layer 416 thereby enhancing the cooling effect to this layer 416. The user may acutely sense this cooling effect when he rubs the layer 416 against other body parts, such as his forehead or face. Accordingly, having apertures 66 in both layers 52 and 53 may enhance the overall cooling effect to the user.

The apparatus 50 may be similarly disposed between fabrics of other articles of clothing to provide cooling sensations to other parts of the body. For example, the apparatus 50 may be embedded within shirts, pants, hats, head bands, and/or other articles of clothing. In addition, the material of the layers 52 and 53 may be composed of conventional clothing fabric material, such as cotton, polyester, wool, etc., such that the apparatus 50 is formed by the article of clothing being worn. As an example, a shirt may comprise an inner layer of clothing fabric and an outer layer of clothing fabric. An inlet may allow a cooling medium to be injected between the two layers of fabric thereby cooling each of the layers. Either of the layers may have vents to allow the cooling medium to egress from the layers of fabric. Other types of articles of clothing may be similarly configured.

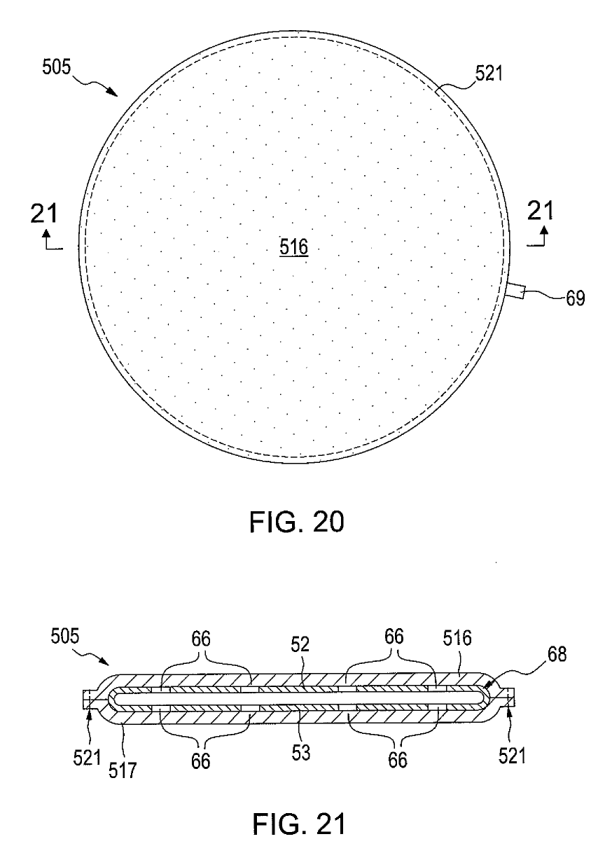

FIGS. 20 and 21 depict an exemplary fabric 505 that can be used to form just about any article of clothing. Similar to the wrist band 411 depicted by FIG. 15, the fabric 505 has two layers 516 and 517 of fabric material, such as cotton, for example, with a medium transfer apparatus 50 embedded between the two layers 516 and 517. The fabric layers 516 and 517 are sewn together along a seam 521.

The base 68 of the apparatus 50 between the fabric layers 516 and 517 may have any shape or size. Preferably, the base 68 is dimensioned to fit between the fabric layers 516 and 517. However, it is possible for at least portions of the base 68 to extrude from the fabric 505. As shown by FIG. 20, the inlet 69 of the apparatus 50 is exposed to allow a medium injection apparatus 110 to be detachably coupled to the inlet 69 so that cooling medium can be injected into the apparatus 50 through the inlet 69 as described above in the previous embodiments.

At least one aperture 66 exists in at least one of the layers 52 and 53 of the apparatus 50 so that the injected cooling medium is directed toward and strikes at least one of the fabric layers 516 and 517. As described above, directing the cooling medium toward a fabric layer in contact with a user's body generally enhances the cooling sensation felt by the user. If desired, apertures 66 may exist in both layers 52 and 53 so that cooling medium is directed toward and strikes both fabric layers 516 and 517. Note that the seam 521 is preferably not airtight so that portions of the cooling medium can escape between the layers 516 and 517.

The fabric 505 of FIG. 20 is generally circular and is dimensioned such that it can fit within an exemplary hat, as will be described in more detail hereafter. However, the fabric 505 may have other shapes and dimensions in other embodiments. Indeed, the fabric 505 may be used to form a shirt, pants, or some other article of clothing. The fabric 505 may be used to form a head strap within a military helmet or some other type of clothing strap. In one embodiment, the inner or outer surface of a canteen may be lined with the fabric 505 so that the fabric 505 can be used to cool the contents of the canteen. The fabric 505 may have a rectangular or some other elongated shape so that it can be wrapped around the neck of a user, like a towel. The inner surface of a military flak jacket may be lined with the fabric 505. In each of the aforedescribed examples, a cooling medium can be injected into the apparatus 50 by a medium injection apparatus 110 in order to cool one or more of the fabric layers 516 and 517 and to provide a cooling sensation to the user. Further, like the insole 85 described above, the cooling of either of the layers 516 and 517 may be significant such that the cooling effects continue long after the injection has ended.

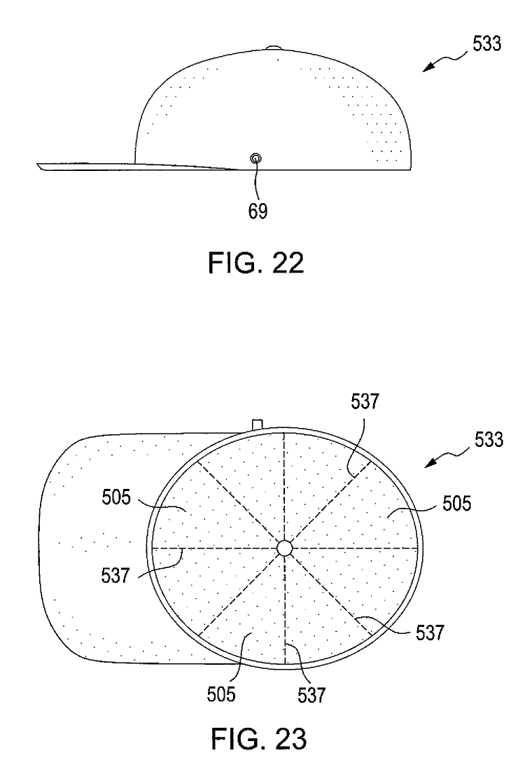

FIG. 22 depicts an exemplary hat 533. As shown by FIG. 23, an inner surface of the hat 533 that would otherwise contact the user's head is lined with the fabric 505 of FIG. 20. Thus, the fabric 505 contacts the user's head and cools the user's head when cooling medium is injected into the apparatus 50. As shown by FIG. 23, the fabric 505 may be sewn to the hat 533 along a plurality of seams 537.

Note that, in some examples, the apparatus 50 can be used without either of the layers 516 or 517. For example, if one of the layers 516 or 517 is to be the only layer directly contacting a user's body, then the other layer 516 or 517 could be eliminated. In such an example, the seam 521 may join the apparatus 50 to one of the layers 516 or 517. Further, in some examples, the apparatus 50 may directly contact the user's body. However, having a layer between the user's body and apparatus 50 is generally desirable to help prevent injury to the user's body due to the temperature of the cooling medium exiting the apertures 66. Further, such a layer may enable longer injections of cooling medium without injury thereby increasing the duration of the cooling effects from an injection. In this regard, the layer between the apparatus 50 and the user's body can be cooled to a very low temperature thereby helping to prolong the effects of the injection. Further, as described above, a heating medium instead of a cooling medium may be injected into the apparatus 50 for any of the aforedescribed embodiments in order to generate a heating sensation rather than a cooling sensation.

In another embodiment of the present disclosure, the cooling medium is directed to an inner layer of an ankle brace such that the ankle brace, and the user's ankle, is significantly cooled by the cooling medium being injected into the inner layer. The cooled ankle brace continues to draw heat from the user's ankle and the user continues to feel a cooling sensation long after the cooling medium injection has ended.

FIG. 24 depicts such an embodiment of the present disclosure. FIG. 24 depicts an ankle brace 2400 from which protrudes an air inlet 2406 from an opening 2401 in the ankle brace 2400. Notably, the ankle brace 2400 fits substantially snug to an ankle (not shown) and a corresponding foot 2409.

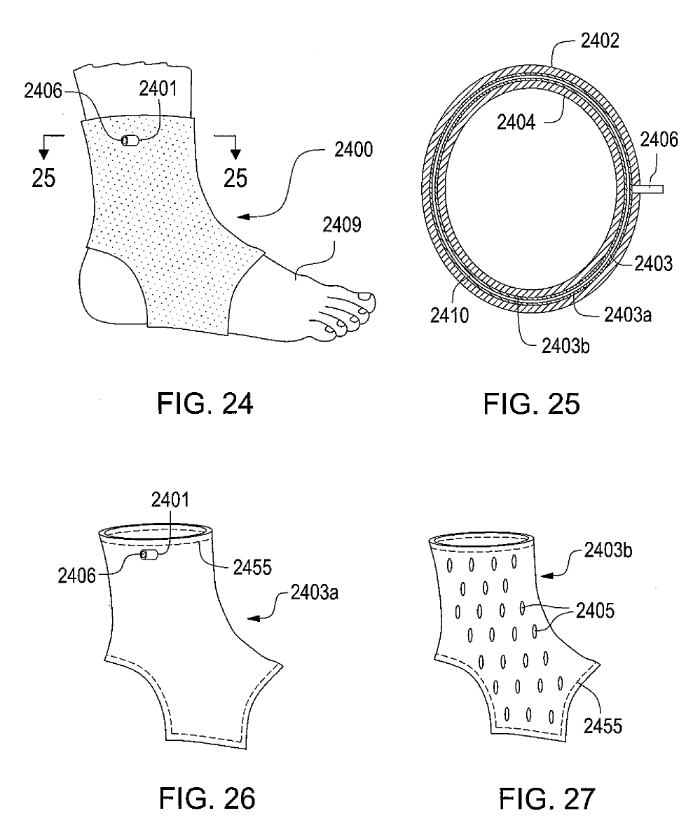

As described hereinabove with reference to FIG. 6, a medium injection apparatus 110 (FIG. 6) is inserted into air inlet 2406. When a user (not shown) actuates the medium injection apparatus 110, cool or cold air (not shown) is delivered to the ankle brace 2400, as described further with reference to FIGS. 25-27.

The delivered air works to cool the user's foot 2409 and ankle (not shown). In this regard, a user can cool the foot 2409 and ankle (not shown) while continuing to engage in physical activity or while the user is resting. Notably, once the user injects cool air into the ankle brace 2400, the ankle brace 2400 continues to draw heat from the user's ankle and foot 2409 and the user continues to feel the cooling sensation long after the cooling medium injection has ended.

FIG. 25 depicts a cross-sectional view of the ankle brace 2400 taken along line 25 (FIG. 24). The ankle brace 2400 comprises an outer layer 2402, a medium transfer apparatus 2403, and an inner layer 2404. The outer layer 2402 and the inner layer 2404 form a shell in which the medium transfer apparatus 2403 resides.

In one embodiment, the outer layer 2402 is made of a stretchable material, such as, for example, Neoprene. Other types of elastic materials may be used for the outer layer 2402 in other embodiments. Further, the inner layer 2404 is made of any type of material, including cotton. A material capable of protecting one's skin from extreme temperatures may also be employed.

The medium transfer apparatus 2403 is substantially similar to the apparatus 50 (FIG. 1) as described hereinabove with reference to FIG. 1. Accordingly, in one embodiment the medium transfer apparatus 2403 is comprised of polyvinyl chloride (PVC) and is about 0.006 inches thick. In addition, the material of the medium transfer apparatus 2403 is flexible. The medium transfer apparatus 2403 has an outer layer 2403a and an inner layer 2403b that are melded together to form a cavity 2410. The outer layer 2403a and the inner layer 2403b are described further with reference to FIGS. 26 and 27. As described hereinabove, when the user injects cool air into via the air inlet 2406, the cool air (not shown) enters the cavity 2410.

FIG. 26 depicts a perspective view of the outer layer 2403a of the medium transfer apparatus 2403, and FIG. 27 depicts a perspective view of the inner layer 2403b of the medium transfer apparatus 2403. As noted, the cavity 2410 is formed by the melding of the outer layer 2403a and the inner layer 2403b.

The outer layer 2403a is substantially solid and comprises the air inlet 2406. Whereas, the inner layer comprises a plurality of apertures 2405. In one embodiment, the outer layer 2403a is melded with the inner layer 2403b to form a bead 2455 joining the outer layer 2403a and inner layer 2403b. The outer and inner layers 2403a and 2403b, as well as the bead 2455, form the cavity 2410 that is substantially airtight except for the plurality of apertures 2405 (FIG. 27) in the inner layer 2403b and the inlet 2406 (FIG. 26).

The medium transfer apparatus 2403 is then inserted between the outer layer 2402 (FIG. 25) and the inner layer 2404 (FIG. 25) of the ankle brace 2400. Such insertion may be accomplished by, for example, sewing the outer layer 2402 to the inner layer 2404 thereby encapsulating the medium transfer apparatus 2403.

When the ankle brace 2400 is assembled, the inlet 2406 protrudes through the opening 2401. Such inlet 2406 allows a medium, such as cooled or heated air, to enter the cavity 2410, and the apertures 2405 allow the medium to exit the cavity 2410.

In another embodiment of the present disclosure, the cooling medium is directed to an inner layer of a knee brace such that the knee brace, and the user's leg, is significantly cooled by the cooling medium being injected into an inner layer of the knee brace. The cooled knee brace continues to draw heat from the user's leg and the user continues to feel a cooling sensation long after the cooling medium injection has ended.

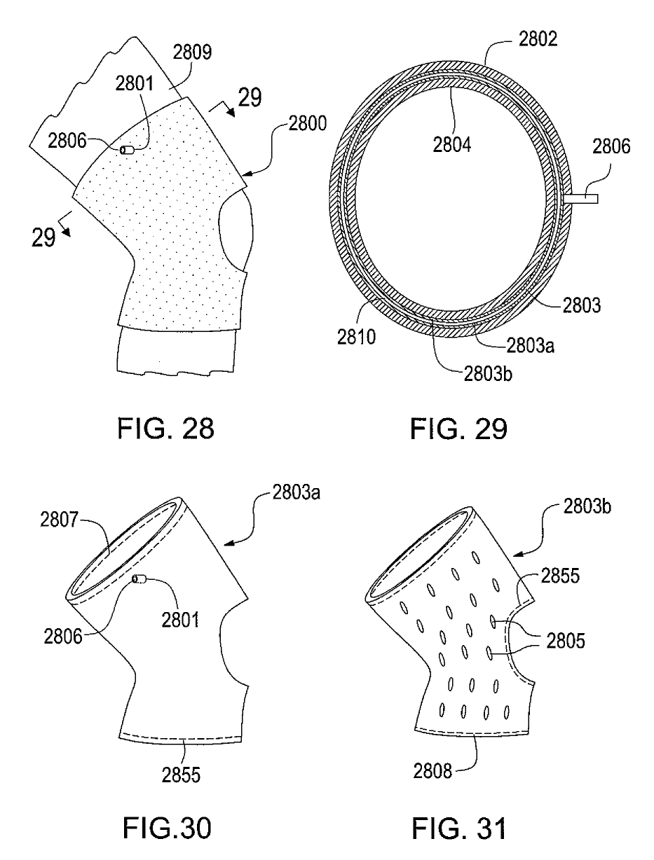

FIG. 28 depicts such an embodiment of the present disclosure. FIG. 28 depicts a knee brace 2800 from which protrudes an air inlet 2806 from an opening 2801 in the knee brace 2800. Notably, the knee brace 2800 fits substantially snug to a leg 2809.

As described hereinabove with reference to FIG. 6, a medium injection apparatus 110 (FIG. 6) is inserted into air inlet 2806. When a user (not shown) actuates the medium injection apparatus 110, cool or cold air (not shown) is delivered to the knee brace 2800, as described further with reference to FIGS. 29-31.

The delivered air works to cool the user's leg 2809. In this regard, a user can cool the leg 2809 while continuing to engage in physical activity or while the user is resting. Notably, once the user injects cool air into the knee brace 2800, the knee brace 2800 continues to draw heat from the user's leg 2809, and the user continues to feel the cooling sensation long after the cooling medium injection has ended.

FIG. 29 depicts a cross-sectional view of the knee brace 2800 taken along line 29 (FIG. 28). The knee brace 2800 comprises an outer layer 2802, a medium transfer apparatus 2803, and an inner layer 2804. The outer layer 2802 and the inner layer 2804 form a shell in which the medium transfer apparatus 2803 resides.

In one embodiment, the outer layer 2802 is made of a stretchable material, such as, for example, Neoprene. Other types of elastic materials may be used for the outer layer 2802 in other embodiments. Further, the inner layer 2804 is made of any type of material, including cotton. A material capable of protecting one's skin from extreme temperatures, for example Thinsulate or Gortex, may also be employed.

The medium transfer apparatus 2803 is substantially similar to the apparatus 50 (FIG. 1) as described hereinabove with reference to FIG. 1, except that the apparatus 2803 is shaped in accordance with a leg and corresponding knee. Accordingly, in one embodiment the medium transfer apparatus 2803 is comprised of polyvinyl chloride (PVC) and is about 0.006 inches thick. In addition, the material of the medium transfer apparatus 2803 is flexible.

The medium transfer apparatus 2803 has an outer layer 2803a and an inner layer 2803b that are melded together to form a cavity 2810. The outer layer 2803a and the inner layer 2803b are described further with reference to FIGS. 30 and 31. As described hereinabove, when the user injects cool air into via the air inlet 2806, the cool air (not shown) enters the cavity 2810.

FIG. 30 depicts a perspective view of the outer layer 2803a of the medium transfer apparatus 2803, and FIG. 31 depicts a perspective view of the inner layer 2803b of the medium transfer apparatus 2803. As noted, the cavity 2810 is formed by the melding of the outer layer 2803a and the inner layer 2803b.

The outer layer 2803a is substantially solid and comprises the air inlet 2806. Whereas, the inner layer comprises a plurality of apertures 2805. In one embodiment, the outer layer 2803a is melded with the inner layer 2803b to form a bead 2855 joining the outer layer 2803a and inner layer 2803b. The outer and inner layers 2803a and 2803b, as well as the bead 2855, form the cavity 2810 that is substantially airtight except for the plurality of apertures 2805 (FIG. 31) in the inner layer 2803b and the inlet 2806 (FIG. 30).

The medium transfer apparatus 2803 is then inserted between the outer layer 2802 (FIG. 29) and the inner layer 2804 (FIG. 29) of the knee brace 2800. Such insertion may be accomplished by, for example, sewing the outer layer 2802 to the inner layer 2804 thereby encapsulating the medium transfer apparatus 2803.

When assembled the inlet 2806 protrudes through the opening 2801. Such inlet 2806 allows a medium, such as cooled or heated air, to enter the cavity 2810, and the apertures 2805 allow the medium to exit the cavity 2810 thereby cooling the leg 2809.

In another embodiment of the present disclosure, the cooling medium is directed to an inner layer of an elbow brace such that the elbow brace, and the user's arm, are significantly cooled by the cooling medium being injected into an inner layer of the elbow brace. The cooled elbow brace continues to draw heat from the user's arm and the user continues to feel a cooling sensation long after the cooling medium injection has ended.

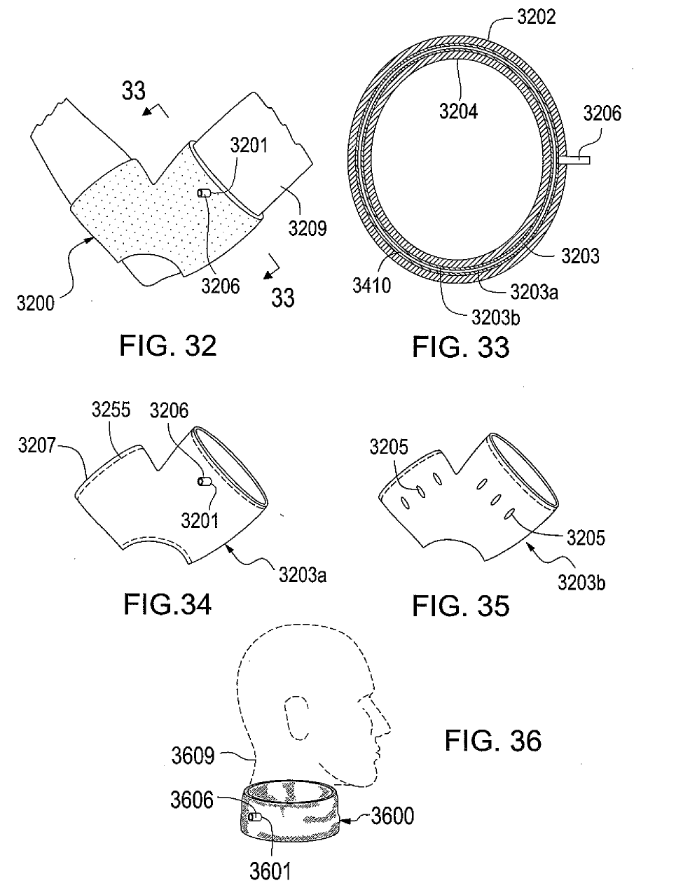

FIG. 32 depicts such an embodiment of the present disclosure. FIG. 32 depicts an elbow brace 3200 from which protrudes an air inlet 3206 from an opening 3201 in the elbow brace 3200. Notably, the elbow brace 3200 fits substantially snug to an arm 3209.

As described hereinabove with reference to FIG. 6, a medium injection apparatus 110 (FIG. 6) is inserted into air inlet 3206. When a user (not shown) actuates the medium injection apparatus 110, cool or cold air (not shown) is delivered to the elbow brace 3200, as described further with reference to FIGS. 33-35.

The delivered air works to cool the user's arm 3209. In this regard, a user can cool the arm 3209 while continuing to engage in physical activity or while the user is resting. Notably, once the user injects cool air into the elbow brace 3200, the elbow brace 3200 continues to draw heat from the user's arm 3209, and the user continues to feel the cooling sensation long after the cooling medium injection has ended.

FIG. 33 depicts a cross-sectional view of the knee brace 3200 taken along line 33 (FIG. 32). The elbow brace 3200 comprises an outer layer 3202, a medium transfer apparatus 3203, and an inner layer 3204. The outer layer 3202 and the inner layer 3204 form a shell in which the medium transfer apparatus 3203 resides.

In one embodiment, the outer layer 3202 is made of a stretchable material, such as, for example, Neoprene. Other types of elastic materials may be used for the outer layer 3202 in other embodiments. Further, the inner layer 3204 is made of any type of material, including cotton. A material capable of protecting one's skin from extreme temperatures, for example Thinsulate or Gortex, may also be employed.

The medium transfer apparatus 3203 is substantially similar to the apparatus 50 (FIG. 1) as described hereinabove with reference to FIG. 1, except that the apparatus 3203 is shaped in accordance with an arm and corresponding elbow. Accordingly, in one embodiment the medium transfer apparatus 3203 is comprised of polyvinyl chloride (PVC) and is about 0.006 inches thick. In addition, the material of the medium transfer apparatus 3203 is flexible.

The medium transfer apparatus 3203 has an outer layer 3203a and an inner layer 2803b that are melded together to form a cavity 3210. The outer layer 3203a and the inner layer 3203b are described further with reference to FIGS. 34 and 35. As described hereinabove, when the user injects cool air into via the air inlet 3206, the cool air (not shown) enters the cavity 3210.

FIG. 34 depicts a perspective view of the outer layer 3203a of the medium transfer apparatus 3203, and FIG. 35 depicts a perspective view of the inner layer 3503b of the medium transfer apparatus 3203. As noted, the cavity 3210 is formed by the melding of the outer layer 3203a and the inner layer 3203b.

The outer layer 3203a is substantially solid and comprises the air inlet 3206. Whereas, the inner layer comprises a plurality of apertures 3205. In one embodiment, the outer layer 3203a is melded with the inner layer 3203b to form a bead 3255 joining the outer layer 3203a and inner layer 3203b. The outer and inner layers 3203a and 3203b, as well as the bead 3255, form the cavity 3210 that is substantially airtight except for the plurality of apertures 3205 FIG. 35) in the inner layer 3203b and the inlet 3206 (FIG. 34).

The medium transfer apparatus 3203 is then inserted between the outer layer 3202 (FIG. 33) and the inner layer 3204 (FIG. 33) of the elbow brace 3200. Such insertion may be accomplished by, for example, sewing the outer layer 3202 to the inner layer 3204 thereby encapsulating the medium transfer apparatus 3203.

When assembled, the inlet 3206 protrudes through the opening 3201. Such inlet 3206 allows a medium, such as cooled or heated air, to enter the cavity 3210, and the apertures 3205 allow the medium to exit the cavity 3210 thereby cooling the leg 3209.

In another embodiment of the present disclosure, the cooling medium is directed to an inner layer of a neck brace such that the neck brace, and the user's neck, are significantly cooled by the cooling medium being injected into an inner layer of the neck brace. The cooled neck brace continues to draw heat from the user's neck and the user continues to feel a cooling sensation long after the cooling medium injection has ended.

FIG. 36 depicts a neck brace 3600 in accordance with such an embodiment of the present disclosure. As described hereinabove with reference to FIG. 6, a medium injection apparatus 110 (FIG. 6) is inserted into an air inlet 3606. When a user (not shown) actuates the medium injection apparatus 10, cool or cold air (not shown) is delivered to the elbow brace 3600 similar to injection of the cool or cold air into the wrist band 411 (FIG. 15), the ankle brace 2400 (FIG. 24), the knee brace 2800 (FIG. 28), and the elbow brace 3200 (FIG. 32).

The delivered air works to cool a user's neck 3609. In this regard, a user can cool the neck 3609 while continuing into physical activity or while the user is resting. Notably, once the user injects cool air into the neck brace 3600, the neck brace 3600 continues to draw heat from the user's arm 3609, and the user continues to feel the cooling sensation long after the cooling medium injection has ended.

For brevity, a cross-section of the neck brace 3600 is not further described. However, the cross-section of the neck brace 3600 would be identical to the cross-section of the wrist band 411 described with reference to FIG. 16, the cross-section of the ankle brace 2400 described with reference to FIG. 25, the cross-section of the knee brace 2800 described with reference to FIG. 29, and the cross-section of the elbow brace 3200 described with reference to FIG. 33. Furthermore, the neck brace 3600 operates identical to such other embodiments, as described hereinabove.

What is claimed is:

1. A device, comprising:
a shell having a first layer and a second layer; and
a medium transfer apparatus positioned between the first layer and the second layer, wherein the medium transfer apparatus is detachably coupled to a medium injection apparatus containing a medium such that the medium is forced by the medium injection apparatus through the medium transfer apparatus thereby altering a temperature of the shell.

2. The device of claim 1, wherein the shell is formed to be worn on a knee.

3. The device of claim 1, wherein the shell is formed to be worn on an ankle.

4. The device of claim 1, wherein the shell is formed to be worn on an elbow.

5. The device of claim 1, wherein the medium transfer apparatus has a first side and a second side and at least one side has a plurality of apertures through which the medium flows.

6. The device of claim 1, wherein the medium transfer apparatus comprises an air inlet that protrudes from at least one of the layers for coupling to the medium injection apparatus.

7. The device of claim 1, wherein at least one of the layers is comprised of polyvinyl chloride.

8. The device of claim 1, wherein the shell is a wristband.

9. The device of claim 1, wherein the shell is formed to be worn on a head.

10. The device of claim 1, wherein the shell is formed to be worn around a neck.

11. The device of claim 1, wherein the medium injection apparatus comprises a spray can.

12. A method, comprising the steps of:
detachably coupling a medium injection apparatus to a medium transfer apparatus that is positioned within a shell, the medium injection apparatus having a medium that is compressed at a pressure greater than atmospheric pressure; and
injecting the medium from the medium injection apparatus through the medium transfer apparatus to the shell.

13. The method of claim 12, further comprising the step of coupling the shell to a wrist.

14. The method of claim 12, further comprising the step of coupling the shell to a head.

15. The method of claim 12, further comprising the step of coupling the shell to a knee.

16. The method of claim 12, further comprising the step of coupling the shell to an elbow.

17. The method of claim 12, further comprising the step of coupling the shell to a neck.

18. The method of claim 12, further comprising the step of coupling the shell to an ankle.

19. The method of claim 12, wherein the medium injection apparatus is a spray can.

20. A device, comprising:
a shell having a first layer an a second layer; and
means positioned between the first layer and the second layer for transferring a medium to the shell thereby altering a temperature of the shell when a medium injection apparatus is coupled to the means.
